(19) United States
(12) Patent Application Publication
(10) Pub. No.: US 2011/0213264 A1
(43) Pub. Date: Sep. 1, 2011

(54) SENSOR ON NON-SEALING PORTION OF TRACHEAL TUBE CUFF

(75) Inventors: Alan Finneran, Tullamore (IE); Garret Coady, Palmerstown (IE); John Desmond, The Curragh (IE); Mark Cleary, Dundrum (IE); David Powell, Dublin (IE); Patrick Dowling, Athy (IE)

(73) Assignee: Nellcor Puritan Bennett LLC, Boulder, CO (US)

(21) Appl. No.: 12/713,351

(22) Filed: Feb. 26, 2010

(51) Int. Cl. A61M 16/04; A61B 5/08

(52) U.S. Cl. 600/532; 128/207.14

(57) ABSTRACT

Various embodiments of a tracheal tube having a sensor disposed on a non-sealing portion of a cuff are provided. Certain embodiments of the tracheal tube may be capable of deploying the sensor during intubation to sense one or more indicators of blood flow characteristics, such as a level of blood gases and/or blood analytes, in the respiratory tract. The sensor on the cuff may be configured to deploy upon inflation of the cuff and to return to its predeployment position upon deflation of the cuff. The sensor may be further adapted to abut the tracheal mucosa of a patient or not contact the tracheal wall at all during deployment.

### BACKGROUND

The present disclosure relates generally to medical devices and, more particularly, to airway devices, such as tracheal tubes.

This section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

Tracheal tubes are often placed in the airway of a patient in medical situations that necessitate protection of the airway from possible obstruction or occlusion. For instance, tracheal tubes may be used in emergency situations, such as when a patient experiences cardiac or respiratory arrest. The underlying condition that necessitates intubation of the patient may also cause a drop in aortic pressure, leading to low blood flow to non-critical organs, such as the respiratory tract, to compensate for an increased need for blood flow to critical organs, such as the brain. A decrease in blood flow to the respiratory tract may be detected by assessing the level of blood gases and/or blood analytes present in the tracheal mucosa.

Some traditional systems measure the level of blood gases and/or blood analytes in the respiratory tract by introducing a sensor into the trachea and contacting the tracheal mucosa. However, critically ill patients are already intubated with a tracheal tube, and an introduction of an additional sensing device can be uncomfortable and burdensome.

Accordingly, systems that deploy the sensor from the tracheal tube already in place in the respiratory tract have been developed. However, such systems often fall short of expectations since they may compromise one or more of the functions of the tracheal tube. For example, some traditional systems may compromise the sealing properties of the cuff coupled to the tracheal tube. Accordingly, there exists a need for improved systems that measure blood gases and/or blood analytes in the respiratory tract without interrupting the proper functioning of the tracheal tube.

### BRIEF DESCRIPTION OF THE DRAWINGS

Advantages of the disclosed techniques may become apparent upon reading the following detailed description and upon reference to the drawings in which:

FIG. 1 is an elevational view of an exemplary endotracheal tube with a sensor located on a non-sealing portion of a tapered cuff in accordance with aspects of the present disclosure;

FIG. 2 is an elevational view of an exemplary endotracheal tube with a sensor located on a non-sealing portion of a tapered cuff in accordance with aspects of the present disclosure;

FIG. 3 illustrates the endotracheal tube of FIG. 1 positioned in a trachea of a patient lying in a semirecumbent position with a deployed sensor in accordance with aspects of the present disclosure;

FIG. 4 is an elevational view of an exemplary endotracheal tube having a sealing cuff and a deployment cuff having a sensor located on a non-sealing portion of the deployment cuff in accordance with aspects of the present disclosure;

FIG. 5 is an elevational view of an exemplary endotracheal tube having a sealing cuff and a deployment cuff having a sensor located on the deployment cuff in accordance with aspects of the present disclosure; and

FIG. 6 illustrates the endotracheal tube of FIG. 4 positioned in a trachea of a patient lying in a semirecumbent position with a deployed sensor in accordance with aspects of the present disclosure.

### DETAILED DESCRIPTION OF SPECIFIC EMBODIMENTS

One or more specific embodiments of the present techniques will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

As described in detail below, embodiments of an endotracheal tube (ETT) having a sensor disposed on a non-sealing portion of a cuff are provided. The ETT may include a lumen in which one or more support cables may be positioned to facilitate a bidirectional exchange of data, power, and so forth between the sensor and an external support system. The tracheal tube may be disposable rather than reusable, capable of sensing one or more indicators of blood flow characteristics, capable of conveying gas to and from a patient, and capable of deploying one or more sensors during intubation. During use, the sensor on the cuff may be configured to deploy upon inflation of the cuff and adapted to return to its predeployment position upon deflation of the cuff. Furthermore, when deployed, the sensor may be adapted to abut the tracheal mucosa of a patient or not contact the tracheal wall at all. Nevertheless, the sensor is configured to measure a presence or amount of at least one blood gas and/or blood analyte, such as carbon dioxide, oxygen, or pH, in the trachea during deployment. In this way, embodiments of the disclosed ETT may be used to indirectly monitor the cardiac state of a patient by monitoring the level of blood gases and/or blood analytes in the respiratory tract. That is, measurements of such gas and analyte levels in the trachea may be used to determine parameters relating to cardiac output, such as blood flow, and may provide insight into possible cardiac pathologies, such as perfusion failure.

The devices and techniques provided herein may enable the ability to sense blood gases and or blood analyte levels while maximizing the sealing capabilities of the cuff because the sensor is associated with the non-sealing portion of the cuff. That is, while the sensor may be disposed on or coupled to the cuff, the placement of the sensor is such that deployment of the sensor does not affect the seal between the cuff and the tracheal wall. For example, in certain embodiments, the cuff may be a tapered cuff, and the sensor may be located on the tapered portion of the cuff, which is not adapted to seal against the tracheal wall. The tapered portion of the cuff may be located toward the proximal or distal end of the tracheal tube, and the sensor may be appropriately positioned so as not to interfere with the seal between the cuff and the trachea. In additional embodiments, the tracheal tube may include a sealing cuff and a deployment cuff, and the sensor may be coupled to the deployment cuff. The foregoing features may have the effect of maintaining the functionalities of traditional tracheal tubes (e.g., providing an unobstructed airway path) while endowing the tracheal tubes with new functionalities (e.g., measuring blood gases and/or blood analytes).

It should be noted that the provided tracheal tubes and methods of operating the tracheal tubes may be used in conjunction with auxiliary devices, such as airway accessories, ventilators, humidifiers, and so forth, which may cooperate with the tracheal tubes to maintain airflow to the lungs of the patient. For instance, the tracheal tubes may be placed in the trachea and coupled to a ventilator to protect the airway from possible obstruction or occlusion in emergency situations, such as when a patient experiences cardiac or respiratory arrest, For further example, the tracheal tubes may be coupled to an interface circuit and/or a monitor that is configured to receive data from the sensor, process such data, and display the processed data to an end user (e.g., medical technician, doctor, nurse, etc.).

Furthermore, although the embodiments of the present invention illustrated and described herein are discussed in the context of endotracheal tubes, it should be noted that presently contemplated embodiments may include a sensor located on a non-sealing portion of the cuff associated with any of a variety of suitable airway devices. For example, the sensor may be coupled to the non-sealing portion of a cuff associated with a tracheostomy tube, a Broncho-Cath™ tube, a specialty tube, or any other airway device with a cuff. Indeed, any device with a cuff designed for use in an airway of a patient may include a sensor disposed on the non-sealing portion of the cuff. Furthermore, as used herein, the term “tracheal tube” may include an endotracheal tube, a tracheostomy tube, a Broncho-Cath™ tube, a specialty tube, or any other airway device.

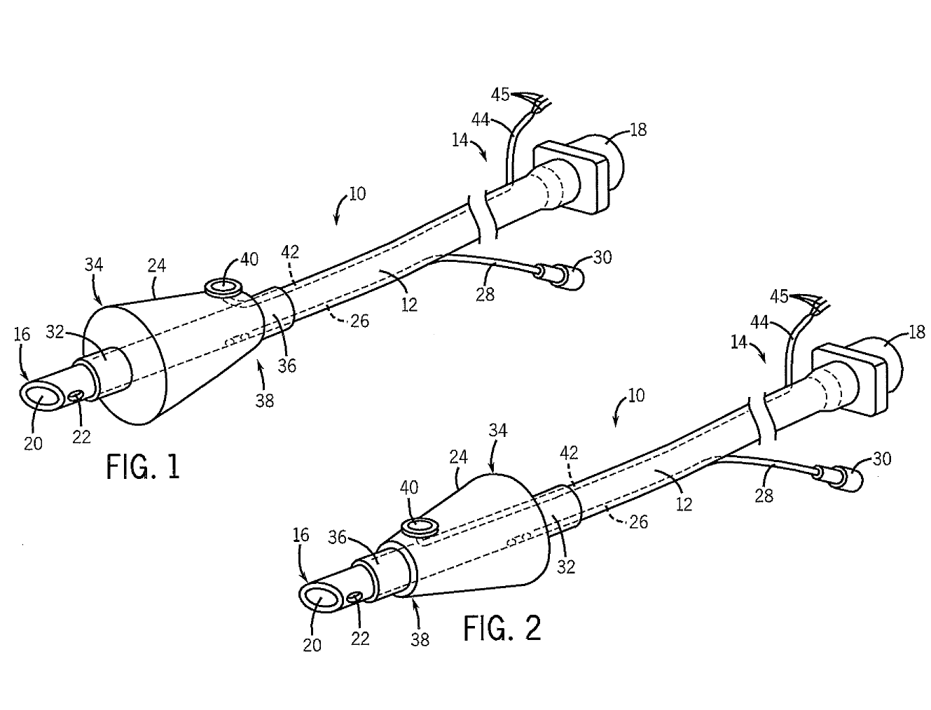

Turning now to the drawings, FIG. 1 is an elevational view of an exemplary ETT 10 in accordance with aspects of the present disclosure. The endotracheal tube 10 includes a central tubular body 12 with proximal and distal ends 14 and 16, respectively. In the illustrated embodiment, the proximal end 14 is outfitted with a connector 18 that may be attached to a mechanical ventilator during operation. The distal end 16 terminates in an opening 20 and may be placed in a patient trachea during operation to maintain airflow to and from the patient's lungs. A Murphy's eye 22 may be located on the tubular body 12 opposite the opening 20 to prevent airway occlusion when the tube assembly 10 is improperly placed within the patient trachea.

As illustrated, a tapered cuff 24 configured to be inflated to seal against the walls of a body cavity (e.g., the trachea) may be attached near the distal end 16 of the tubular body 12, or along the body. The cuff 24 may be inflated via an inflation lumen 26 terminating in an inflation tube 28 connected to a fixture 30 located at the proximal end 14 of the tubular body 12. A first shoulder 32 of the tapered cuff 24 secures a non-tapered end 34 of the cuff 24 to the tubular body 12. Likewise, a second shoulder 36 of the cuff 24 attaches a tapered end 38 of the cuff 24 to the tubular body 12, In some embodiments, the first shoulder 32 and/or the second shoulder 36 may be folded up inside the cuff 24.

The tubular body 12 and the cuff 24 may be formed from materials having desirable mechanical properties (e.g., puncture resistance, pin hole resistance, tensile strength, and so forth) and desirable chemical properties (e.g., biocompatibility). In one embodiment, the walls of the cuff 24 may be made of a polyurethane (e.g., Dow Pellethane® 2363-80A) having suitable mechanical and chemical properties. In other embodiments, the walls of the cuff 24 may be made of a suitable polyvinyl chloride (PVC). In certain embodiments, the cuff 24 may be generally sized and shaped as a high volume, low pressure cuff that may be designed to be inflated to pressures between about 15 cm and 30 cm of water.

A sensor 40 is disposed on a non-sealing portion of the cuff 24. That is, the sensor may be located anywhere on the cuff 24 that is not configured to provide the seal desired between the body of the tube and the body tissues (e.g., directly contact the body cavity, such as the tracheal wall), during inflation of the cuff 24. For instance, in one embodiment, the sensor may be positioned at the tapered end 38 of the cuff as shown in FIG. 1. The sensor 40 is connected to a dedicated lumen 42 terminating in a conduit 44 and one or more conductors 45. That is, one or more support cables and/or conductors may be positioned in the lumen 42 to facilitate a bidirectional exchange of data, power, and so forth between the sensor 40 and an external support system. For example, a printed conductor or a conductive polymer may be located on the outside or the inside of a wall of the lumen 42 or may be encapsulated in a wall of the tubular body 12. For further example, during or after intubation, the sensor 40 may be supplied with power and may export data via the lumen 42. In this way, the lumen 42 and the conduit 44 facilitate bidirectional communication between the sensor 40 located within the patient and the support system positioned outside the patient via the conductors.

In the embodiment illustrated in FIG. 1, the tapered end 38 of the cuff 24 with the sensor 40 is positioned toward the proximal end 14 of the tracheal tube 10, and the non-tapered end 34 of the cuff 24 is positioned toward the distal end 16 of the tracheal tube 10. Such a positioning of the tapered cuff 24 may offer distinct advantages over traditional tapered cuffs that feature the tapered end of the cuff located toward the distal end of the tracheal tube. For instance, such a tapered cuff 24 may allow the sensor 40 to be located toward the proximal side of the tracheal tube 10 during intubation, thus simplifying the mechanical design of the tracheal tube and minimizing the length of the lumen 42.

In further embodiments, such as in the embodiment of FIG. 2, the cuff 24 may be reversed in orientation with respect to the tracheal tube 10 of FIG. 1. That is, as shown, the tapered end 38 of the cuff 24 and the second shoulder 36 are located toward the distal end 16 of the tracheal tube 10. The non-tapered end 34 of the cuff 24 and the first shoulder 32 are located toward the proximal end 14 of the tracheal tube 10. In such an embodiment, the sensor 40 is still positioned on the non-sealing portion (e.g., the tapered end 38) of the cuff 24. However, as compared to the sensor of the embodiment of FIG. 1, the sensor 40 of FIG. 2 is located toward the distal end 16 of the tracheal tube 10. The lumen 42 of this embodiment extends through the cuff 42 along the tubular body 12 to reach the sensor 40. Positioning the cuff 24 and the sensor 40 in this way may facilitate easy insertion of the tracheal tube 10.

While in the embodiments of FIGS. 1 and 2, a single sensor is located on the non-sealing portion of the cuff 24, in other embodiments any suitable number of sensors may be located in a variety of advantageous positions along or around the non-sealing portion of the cuff 24. For instance, a plurality of sensors and dedicated lumens may be located radially around the tubular body 12 such that measurements may be taken from a variety of radial positions around the airway of the patient, Such measurements may then be compared, averaged or otherwise processed to account for regional fluctuations that may occur in certain areas of the mucosa. For further example, multiple sensors may be located along the length of the non-sealing portion of the cuff 24 in order to acquire measurements at varying depths within the patient trachea. Indeed, any arrangement of any number of sensors positioned on the non-sealing portion of the cuff may be employed.

Furthermore, although the illustrated embodiments show a tapered cuff, further embodiments may feature one or more sensors located on a non-sealing portion of a non-tapered cuff in accordance with aspects of the present invention. In the presently contemplated embodiments, the sensor may contact the tracheal mucosa directly to obtain a blood gas or blood analyte measurement or may obtain the measurement via equilibration with gases or analytes located in the tracheal cavity adjacent the mucosa and/or the tracheal wall tissue. Accordingly, the sensor may be any suitable carbon dioxide, oxygen, pH, or other gas or analyte sensor, such as an electrochemical sensor, a fluorometric sensor, or a mid-infrared sensor. Furthermore, the sensor may be configured to simultaneously or sequentially measure more than one gas or analyte level.

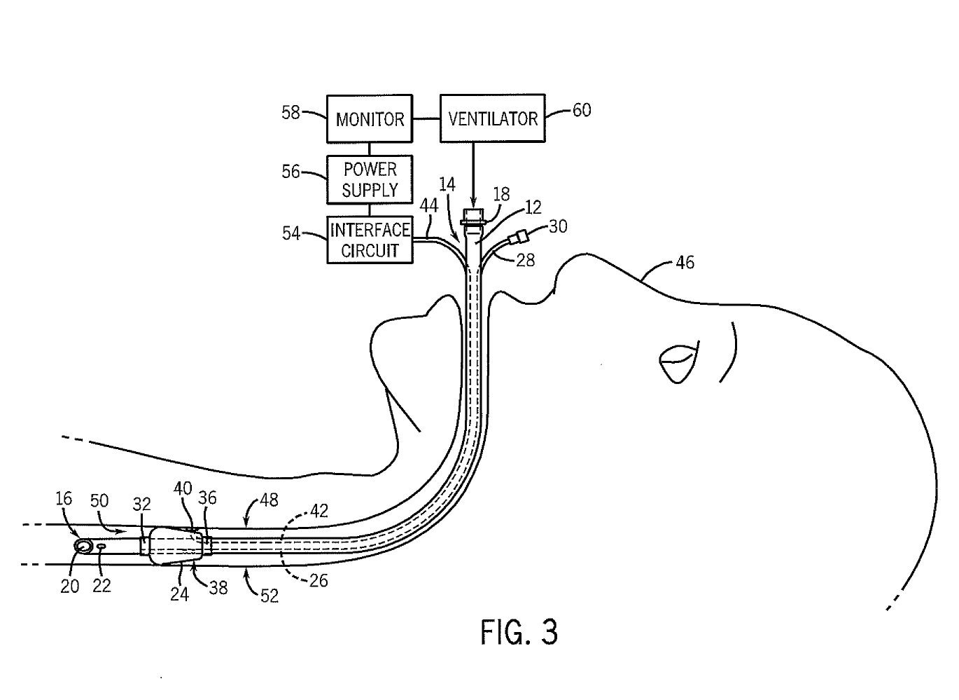

FIG. 3 illustrates an exemplary system including a patient 46 intubated with the endotracheal tube 10 of FIG. 1 in accordance with embodiments of the present invention. As illustrated, the patient 46 is lying in a conventional semirecumbent position as may be typical during long term intubations. In the illustrated embodiment, the sensor 40 is located on the tapered end 38 of the cuff 24 such that the sensor is disposed on the side of the cuff 24 that faces the ventral side of the patient during intubation in the semirecumbent position. That is, in the embodiment shown, the sensor 40 may be located such as to be in contact with a first wall 48 of the trachea 50. When certain sensors 40 are used, this position may offer advantages over positioning close to a second wall 52 of the trachea 50 since mucus may be prone to accumulating near the dorsal side of the patient during intubation in a semirecumbent position. By positioning such sensors 40 near the ventral side of the patient in some embodiments, interference from accumulated secretions may be prevented and inclusion of one or more suctioning ports may be allowed. However, other embodiments may feature one or more sensors 40 that are minimally affected or unaffected by secretion accumulation. In such embodiments, the one or more sensors 40 may be placed in any desirable location around the circumference of the trachea.

As before, the dedicated lumen 42 and conduit 44 may couple one or more devices or systems to the sensor 40 during intubation. That is, the sensor 40 and dedicated lumen 42 may be positioned within the trachea 50 of the patient 46 during intubation while the conduit 44 may be externally located. In the illustrated embodiment, the external conduit 44 is communicatively coupled to an interface circuit 54 that is configured to receive and process measurement data acquired by the sensor 40. The interface circuit 54 is coupled to a power supply 56 that provides power for the sensor 40 and any electronics associated with the sensor 40. The interface circuit 54 may also facilitate the transfer of power to the sensor 40 in some embodiments. The power supply 56 is further coupled to a monitor 58 that is adapted to interpret and display the measurements received from the sensor 40 via the interface circuit 54. To that end, the monitor 58 may include a memory, a display, code configured to provide a specific output, and so forth. For example, the monitor 58 may include software adapted to integrate measurements taken at preset intervals over a predetermined period of time and/or to average or otherwise process measurements taken from multiple positions within the trachea 50. The monitor 58 may be connected to a ventilator 60 that supplies air to the patient 46 through connector 18.

Still further, in other embodiments, the sensor 40 may be adapted to unidirectionally or bidirectionally communicate with one or more external devices via wireless communication. That is, in some embodiments, the sensor 40 may not be coupled to the external devices via the conductors. In such embodiments, the sensor 40 may wirelessly communicate with devices such as a monitor, ventilator, mobile phone, PDA, or central communications point. Further embodiments may feature a single conductor that couples the sensor 40 to the power supply 56, while data communication occurs via a wireless route.

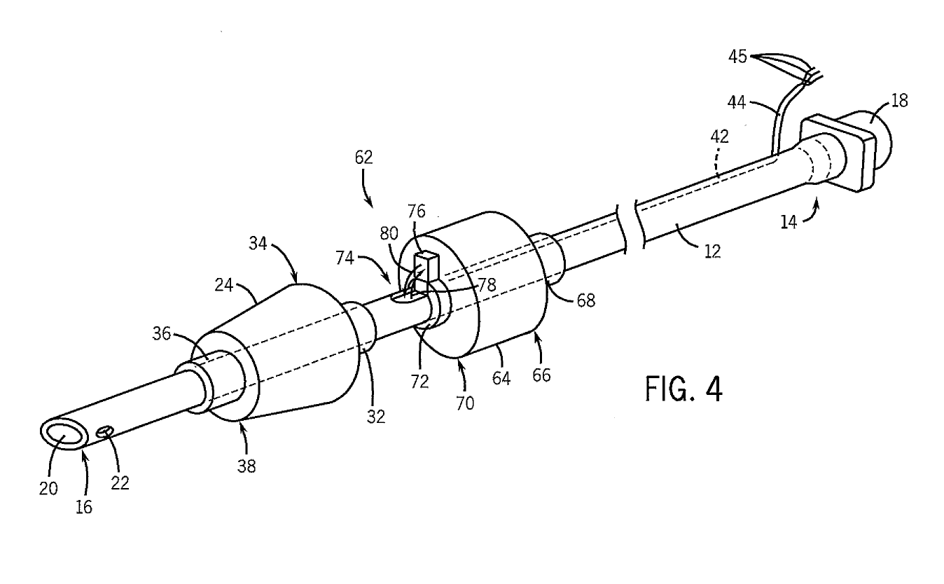

FIG. 4 is an elevational view of an exemplary ETT assembly 62 in accordance with aspects of the present disclosure. The endotracheal tube assembly 62 includes the central tubular body 12 with proximal and distal ends 14 and 16, respectively, as before. The ETT assembly 62 also includes the tapered cuff 24 attached to the distal end 16 of the tubular body 12 and configured to be inflated to seal against the walls of the trachea. As in FIG. 2, the first shoulder 32 of the tapered cuff 24 secures the non-tapered end 34 of the cuff 24 to the tubular body 12 in the direction toward the proximal end 14 of the tubular body 12. Similarly, the second shoulder 36 of the cuff 24 attaches the tapered end 38 of the cuff 24 to the tubular body 12 in the direction toward the distal end 16 of the tubular body 12. However, in contrast to the embodiments of FIGS. 1-3, the tracheal tube 62 of FIG. 4 includes a second cuff 64 with a first end 66 coupled to the tubular body 12 via a third shoulder 68 and a second end 70 coupled to the tubular body 12 with a fourth shoulder 72.

A recess 74 is located in the tubular body 12 between the first shoulder 32 and the fourth shoulder 72. The recess 74 is configured to receive a sensor 76 that is shown in a deployed position in FIG. 4. That is, the recess 74 and the sensor 76 are sized and shaped such that during intubation and extubation of the patient, the sensor 76 rests in the recess 74. To that end, the sensor 76 may be hinged to the tubular body 12 and/or the fourth shoulder 72 such that the sensor 76 rotates into sensing position along the path indicated by arrow 78 and rotates out of sensing position along the path indicated by arrow 80. When in sensing position, as shown, the sensor 76 may be adapted to contact the tracheal mucosa or remain in close proximity to the mucosa for measurement acquisition. Isolation of a cavity located between the sealing cuff 24 and the deployment cuff 64 may facilitate the acquisition of measurements in embodiments in which the sensor 76 is not configured to contact and/or seal against the wall of the trachea mucosa. In such embodiments, the blood gases and/or blood analytes may equilibrate between the mucosa and the isolated cavity. Additionally, the sensor 76 may be coupled to the tubular body 12 at the third shoulder 68 and adapted to rotate into sensing position against the first end 66 of the cuff 64. Nonetheless, during sensing, the sensor 76 deploys to a vertical position against the cuff 64 such that the sealing function of the cuff 64 is not compromised by measurement acquisition.

It should be noted that in further embodiments, the sensor 76 may be coupled to the tubular body 12 at the first shoulder 32 and adapted to rotate into sensing position against the non-tapered end 34 of the cuff 24. In this embodiment, the sensor 76 deploys to a vertical position against the cuff 24 such that the sealing function of the cuff 24 is not compromised by measurement acquisition. In this embodiment, the cuff 64 may still be coupled to the tubular body 12 to create a cavity between cuff 24 and cuff 64 and/or to facilitate sensor alignment when deployed. Still further, in embodiments in which the sensor 76 is coupled to the shoulder 32 and the cuff 24, the second cuff 64 may be eliminated.

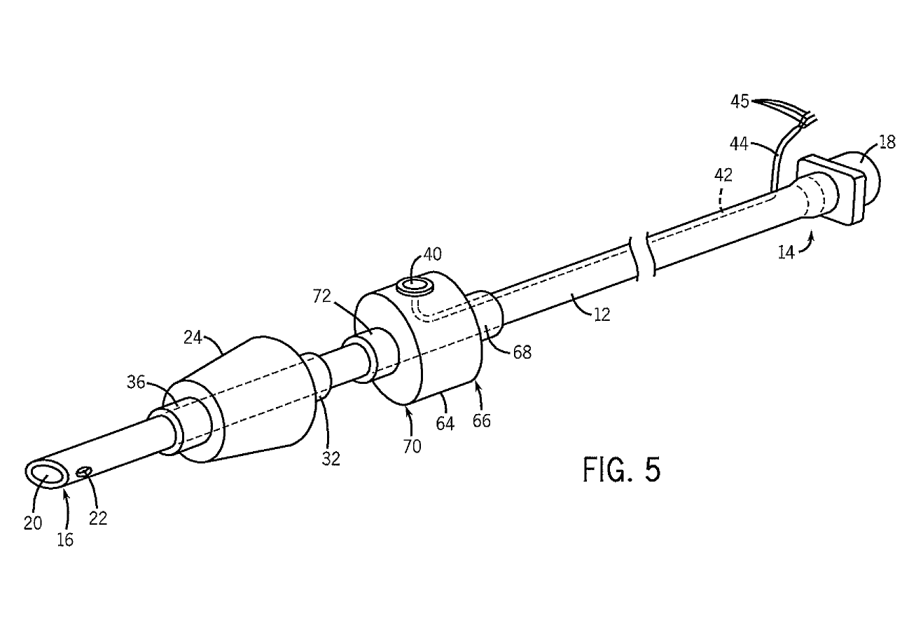

FIG. 5 is an elevational view of an exemplary ETT assembly in accordance with aspects of the present disclosure. The endotracheal tube assembly includes the central tubular body 12 with proximal and distal ends 14 and 16, respectively, as before. The ETT assembly further includes the tapered cuff 24 attached to the distal end 16 of the tubular body 12 and configured to be inflated to seal against the walls of the trachea. As in FIG. 4, the first shoulder 32 of the tapered cuff 24 secures the non-tapered end 34 of the cuff 24 to the tubular body 12 in the direction toward the proximal end 14 of the tubular body 12. Similarly, the second shoulder 36 of the cuff 24 attaches the tapered end 38 of the cuff 24 to the tubular body 12 in the direction toward the distal end 16 of the tubular body 12. As in FIG. 4, the ETT assembly includes the deployment cuff 64 coupled to the tubular body 12 via the third shoulder 68 and the fourth shoulder 72. However, in contrast to FIG. 4, the sensor 40 is located on the deployment cuff 64. That is, the sensor 40 may be located anywhere on the deployment cuff 64 since the sealing cuff 24 is adapted to seal against the tracheal wall and maintain the ETT assembly in the trachea,

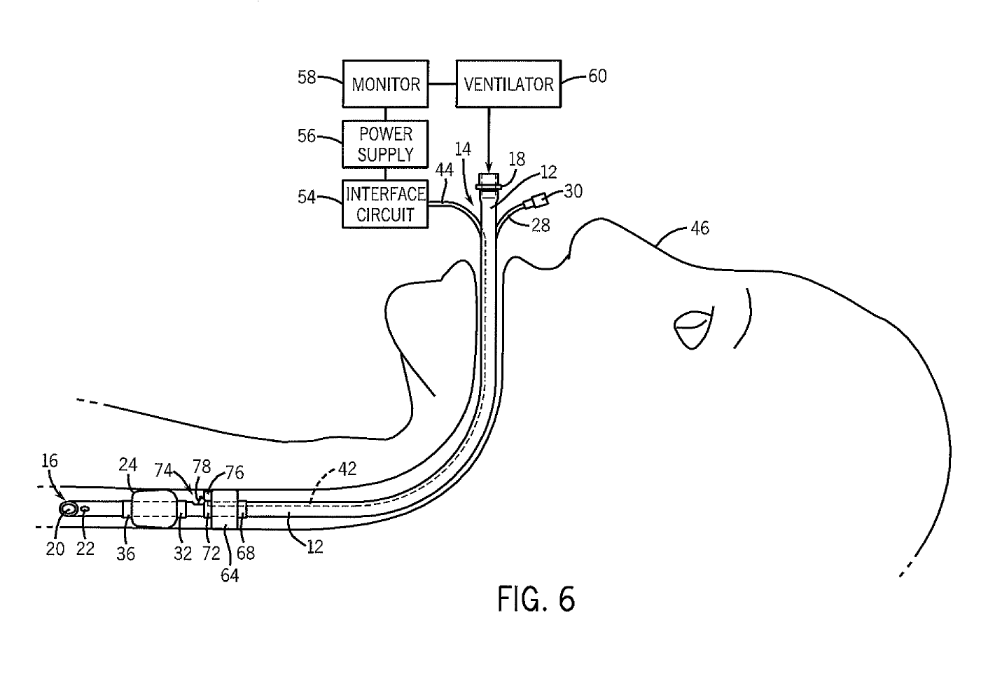

FIG. 6 illustrates an exemplary system including a patient 46 intubated with the endotracheal tube 62 of FIG. 4 in accordance with embodiments of the present invention. As illustrated, the patient 46 is lying in a conventional semirecumbent position as may be typical during long term intubations. During deployment, as shown, the sensor 76 is located adjacent the ventral side of the patient during intubation in the semirecumbent position. Since this embodiment relies on proper rotation of the sensor 76 into position in order to effectively acquire measurements, such a positioning may be advantageous. That is, since mucus may be prone to accumulating near the dorsal side of the patient during intubation in a semirecumbent position, positioning of the sensor 76 near the ventral side of the patient may limit interference from accumulated mucus.

As before, the sensor 76 is connected to the dedicated lumen 42 terminating in the conduit 44 and support cables may be positioned in the lumen 42 to facilitate bidirectional communication between the sensor 76 and an externally located support system. The external conduit 44 communicatively couples the interface circuit 54 to the sensor 76. The interface circuit 54 in turn couples the power supply 56 with the sensor 40, In this way, the interface circuit 54 facilitates the transfer of power and data to and from the sensor 76. The power supply 56 is also coupled to the monitor 58 that is adapted to interpret and display the measurements received from the sensor 76 via the interface circuit 54. The monitor 58 is connected to the ventilator 60 that supplies air to the patient 46 through the connector 18.

While the disclosure may be susceptible to various modifications and alternative forms, specific embodiments have been shown by way of example in the drawings and have been described in detail herein. However, it should be understood that the embodiments provided herein are not intended to be limited to the particular forms disclosed. Rather, the various embodiments may cover all modifications, equivalents, and alternatives falling within the spirit and scope of the disclosure as defined by the following appended claims.

What is claimed is:

1. A tracheal tube, comprising:
a tubular body having an open distal end for ventilating a patient;
a cuff disposed around the tubular body above the open distal end and configured to be inflated to seal the cuff against a wall of a patient trachea; and
a sensor coupled to a non-sealing portion of the cuff and configured to measure a level of a blood gas and/or a blood analyte in the trachea.

2. The tracheal tube of claim 1, wherein the cuff is a tapered cuff and the sensor is coupled to a tapered portion of the tapered cuff.

3. The tracheal tube of claim 1, comprising a connector configured to attach to a proximal end of the tubular body to communicatively couple the tracheal tube to a controlled ventilation device.

4. The tracheal tube of claim 1, comprising a lumen extending along the tubular body between a location on the tracheal tube positioned outside the patient when in use to a location on the tracheal tube positioned inside the patient, and terminating near the sensor.

5. The tracheal tube of claim 4, wherein the lumen is configured to receive one or more conductors to communicatively couple the sensor to at least one of a monitor, a ventilator, a power supply, or an interface circuit.

6. The tracheal tube of claim 1, wherein the sensor is radially located toward a ventral portion of the wall of the trachea when the patient is intubated in a semirecumbent position.

7. The tracheal tube of claim 1, comprising an inflation lumen extending along the tubular body between a location on the tracheal tube positioned outside the patient when in use to a location of the cuff positioned inside the patient, wherein the inflation lumen is adapted to deliver inflation gas to the cuff.

8. The tracheal tube of claim 1, comprising a Murphy eye integral with the tubular body and configured to substantially prevent airway occlusion.

9. The tracheal tube of claim 1, wherein the blood gas and/or blood analyte is carbon dioxide, oxygen, pH, or a combination thereof.

10. A tracheal tube, comprising:
a tubular body having an open distal end for ventilating a patient;
a sealing cuff disposed around the tubular body and configured to be inflated to seal the cuff against a wall of a patient trachea; and
a deployment cuff disposed around the tubular body and configured to be inflated to deploy a sensor proximate to the wall of the patient trachea, wherein the sensor is adapted to measure a level of a blood gas and/or a blood analyte in the patient's trachea.

11. The tracheal tube of claim 10, comprising a recess disposed in the tubular body, wherein the recess is configured to receive the sensor during intubation and extubation.

12. The tracheal tube of claim 10, comprising a cavity located between the sealing cuff and the deployment cuff, wherein the sensor is configured to measure the blood gas or blood analyte level in the cavity.

13. The tracheal tube of claim 10, wherein the blood gas and/or blood analyte is carbon dioxide, oxygen, pH, or a combination thereof.

14. The tracheal tube of claim 10, comprising a lumen extending along the tubular body between a location on the tracheal tube positioned outside the patient when in use to a location on the tracheal tube positioned inside the patient and terminating near the sensor.

15. A tracheal tube, comprising:
a tubular body comprising an open distal end for ventilating a patient;
a cuff disposed around the tubular body, having a sealing portion and a non-sealing portion, and being configured to be inflated to seal the sealing portion of the cuff against a wall of the patient trachea;
a sensor disposed on a non-sealing portion of the cuff and adapted to measure a level of a blood gas and/or a blood analyte; and
a lumen extending along the tubular body between a location on the tracheal tube positioned outside the patient when in use to a location on the non-sealing portion of the cuff positioned inside the patient when in use, and terminating near the sensor, wherein one or more conductors are disposed in the lumen and coupled to the sensor.

16. The tracheal tube of claim 15, wherein the cuff is a tapered cuff and the lumen terminates in the sensor located on a tapered end of the tapered cuff.

17. The tracheal tube of claim 15, comprising a suction lumen extending along the tubular body between a location on the tracheal tube positioned outside the patient when in use to a location on the tracheal tube positioned inside the patient, and terminating in a port through which secretions may be aspirated via the suction lumen.

18. The tracheal tube of claim 15, wherein the blood gas and/or blood analyte is carbon dioxide, oxygen, pH, or a combination thereof.

19. The tracheal tube of claim 15, comprising a connector configured to attach to a proximal end of the tubular body to communicatively couple the tracheal tube to a controlled ventilation device.

20. The tracheal tube of claim 15, wherein the conductor is configured to be coupled to at least one of a monitor, a ventilator, a power supply, or an interface circuit.
